(19) United States
(12) Patent Application Publication
(10) Pub. No.: US 2005/0070962 A1
(43) Pub. Date: Mar. 31, 2005

(54) Methods and systems for treating heart failure with vibrational energy

(75) Inventors: Debra S. Echt, Woodside, CA (US); Axel F. Brisken, Fremont, CA (US); Richard E. Riley, Palo Alto, CA (US); Mark W. Cowan, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EBR Systems, Inc., Mountain View, CA (US)

(21) Appl. No.: 10/869,631

(22) Filed: Jun. 15, 2004

Related U.S. Application Data
(60) Provisional application No. 60/507,719, filed on Sep. 30, 2003.

(51) Int. Cl. A61N 1/362

(52) U.S. Cl. 607/3

(57) ABSTRACT

Methods and apparatus for treating heart failure rely on delivering ultrasonic or other vibrational energy to the heart. The energy may be delivered acutely or chronically, in response to detected cardiac events, in response to manual actuation and/or in response to operation of an implantable defibrillator. The vibrational transducer is implanted so that the vibrational energy can be directed toward at least a portion of the heart in order to increase contractility, vasodilation, tissue perfusion, and/or cardiac output.

### CROSS-REFERENCES TO RELATED APPLICATIONS

The present application claims priority from U.S. Patent Application Ser. No. 60/507,719 (Attorney Docket No. 021629-000300), filed Sep. 30, 2003, the full disclosure of which is incorporated herein by reference.

The disclosure of the present application is also related to the following applications being filed on the same day as the present application: U.S. Patent Ser. No. 10/______ (Attorney Docket No. 021834-000130US); U.S. patent application Ser. No. 10/______ (Attorney Docket No. 021834-000210US); and U.S. patent application Ser. No. 10/______ (Attorney Docket No. 021834-000620US), the full disclosures of which are incorporated herein by reference.

### BACKGROUND OF THE INVENTION

1. Field of the Invention

The present invention relates generally to medical devices and treatment methods. More particularly, the present invention relates to methods and apparatus for treating heart failure and conditions related to heart failure with vibrational energy.

Heart failure (HF) currently affects over five million patients in the United States alone. The number of patients has been steadily increasing due to both aging of the population and the improved ability to extend the life of patients with chronic cardiac conditions. HF is defined by the American College of Cardiology (ACC)/American Heart Association (AHA) Task Force as a complex clinical syndrome characterized by impairment of the ventricle to fill with or eject blood. HF generally results from underlying factors such as hypertension, diabetes, valvular disease, cardiomyopathy, coronary artery disease, and structural changes to the heart muscle. HF is characterized by reduced ventricular wall motion in systole and/or diastole as well as a low ejection fraction. As the heart becomes less able to pump blood, patients develop symptoms of fluid retention, shortness of breath, and fatigue.

While medications have been developed to treat HF, none have been completely effective. It would thus be desirable to provide devices which would be able to stabilize heart function or in some cases improve heart function in patients suffering from or at risk of heart failure.

Therapeutic ultrasound applied to the heart has been reported to increase cardiac contractility, improve cardiac performance, cause coronary vasodilation, and increase myocardial tissue perfusion. The reports describe the acute use of continuous and pulsed application of ultrasound over a wide range of treatment durations, time intervals, frequencies, and intensities.

For these reasons, it would be desirable to provide implantable and/or continuously available apparatus and methods for directing ultrasonic and other vibrational energy to the heart in order to enhance cardiac function as well as provide prophylactic treatment for heart failure.

2. Description of the Background Art

U.S. Pat. No. 4,651,716 describes externally applied ultrasonic energy to enhance cardiac contractility. Patents describing the treatment of heart conditions using mechanical shock therapy include U.S. Pat. Nos. 6,408,205; 6,330,475; 6,110,098; and 5,433,731. Other patents of interest include U.S. Pat. Nos. 6,539,262; 6,439,236; 6,233,484, 5,800,464; 5,871,506; 5,292,338; 5,165,403; and 4,651,716; and WO 03/070323 and WO 99/061058, which relate to other systems applying treatment for arrhythmias, heart failure, and contractility. Medical publications discussing the effects of ultrasound energy and/or mechanical action on the heart and heart failure treatments include:
ACC/AHA Task Force on Practice Guidelines. Evaluation and Management of Chronic Heart Failure in the Adult. JACC 2002;38:2101-13.
Dalecki D, Keller B B, Raeman C H, Carstensen E L. Effects of pulsed ultrasound on the frog heart: I. Thresholds for changes in cardiac rhythm and aortic pressure. Ultrasound in Med. & Biol. 1993; 19:385-390.
Dalecki D, Keller B B, Carstensen E L, Neel D S, Palladino J L, Noordergraaf A. Thresholds for premature ventricular contractions in frog hearts exposed to lithotripter fields. Ultrasound in Med. & Biol. 1991; 17:341-346.
Dalecki D. et al., Effects of pulsed ultrasound on the frog heart: I. Thresholds for changes in cardiac rhythm and aortic pressure. Ultrasound in Med & Biol. 1993;19:385-390.
Dalecki D, Raeman C H, Carstensen E L. Effects of pulsed ultrasound on the frog heart: II. An investigation of heating as a potential mechanism. Ultrasound in Med. & Biol. 1993; 19:391-398.
Feldman A and Bristow M. Comparison of medical therapy, resynchronization and defibrillation therapies in heart failure trial (COMPANION). Presented at ACC 2003 Late Breaking Clinical Trials.
Forester G V et al., Ultrasound Intensity and Contractile Characteristics of Rat Isolated Papillary Muscle. Ultrasound in Med. And Biol. 1985;11(4):591-598
Forester G V, Roy O Z, and Mortimer A J, Enhancement of contractility in rat isolated papillary muscle with therapeutic ultrasound. Mol. Cell Cardiol. 1982; 14(8):475-7.
Franz M R. Mechano-electrical feedback in ventricular myocardium. Cardiovascular Research. 1996; 32:15-24.
Hu H, Sachs F. Stretch-activated ion channels in the heart. J. Mol. Cell Cardiol. 1997; 29:1511-1523.
Kohl P, Hunter P, Noble D. Stretch-induced changes in heart rate and rhythm: clinical observations, experiments and mathematical models. Progress in Biophysics & Molecular Biology. 1999; 71:91-138.
McPherson D and Holland C, Seizing the Science of Ultrasound Beyond Imaging and Into Physiology and Therapeutics. Journal of the American College of Cardiology 2003;41:1628-30.
Meltzer R S, Schwarz K Q, et al. Therapeutic Cardiac Ultrasound. American Journal of Cardiology. 1991;67:422-4
Miyamoto T. et al. Coronary Vasodilation by Noninvasive Transcutaneous Ultrasound An In Vivo Canine Study. Journal of the American College of Cardiology. 2003;41:1623-7
Mortimer A j et al., Letter to the Editor: Altered Myocardial Contractility with Pulsed Ultrasound. Ultrasound in Med and Biol. 1987;13(9):L567-9
Moss A J, Zareba W, Hall W J, Klein H, Wilber D J, Cannom D S, Daubert J P, Higgins S L, Brown M W, Andrews M L. Prophylactic implantation of a defibrillator in patients with myocardial infarction and reduced ejection fraction. N Engl J. Med. 2002; 346:877-933.
Reiter M J. Effects of mechano-electrical feedback: potential arrhythmogenic influence in patients with congestive heart failure. Cardiovascular Research. 1996; 32:44-51.
Suchkova V N, et al., Ultrasound improves tissue perfusion in ischemic tissue through a nitric oxide-dependent mechanism. Throm Haemost. 2002;88:865-70.
Zakharov S I, Bogdanov K Y u, Rosenshtraukh L V. The effect of acoustic cavitation on the contraction force and membrane potential of rat papillary muscle. Ultrasound Med. Biol. 1989; 15 (6):561-5.
Zakharov S I, Bogdanov K Y u, Gavrilov L R, lushin V P, Rozenshtraukh L V. The action of ultrasound on the contraction strength and cation potential of the papillary muscle of the rat heart. Biul Eksp Biol Med. 1989; Apr.; 107(4):423-6.

### BRIEF SUMMARY OF THE INVENTION

The present invention relies on the beneficial and ameliorative effects of vibrational energy to improve cardiac function in patients suffering from or at risk of heart failure. Vibrational energy is applied from an implanted or external transducer under a variety of particular protocols, depending on the patient condition and the desired therapy. In all cases, the delivery of vibrational energy to the heart provides at least one of an increase in contractility, vasodilation, tissue perfusion, and/or an increase in cardiac output.

In a first particular protocol, vibrational energy may be delivered substantially continually in order to promote long-term improvement in cardiac function. By “substantially continually,” it is meant that vibrational energy will be applied at all times or, more usually, at regular intervals in order to promote a long-term improvement in heart function.

In a second particular protocol according to the present invention, the vibrational energy may be delivered in response to a manually initiated signal, typically initiated by medical personnel or the patient in response to an acute cardiac episode in order to treat symptoms, or alternatively, in order to assess whether a patient would benefit from vibrational therapy.

In a third particular protocol according to the present invention, the vibrational energy will be delivered in automatic response to detection of a cardiac event. In such cases, the event will preferably be detected by implanted sensors which are part of or linked to the control circuitry for a vibrational transducer. For example, the sensors would be adapted for use to detect changes in blood pressure, O₂ saturation, heart chamber dimensions, changes or patterns in ECG waveform morphology, contractility, or other types of indicators of heart failure conditions.

In a fourth particular protocol according to the present invention, the vibrational energy will be delivered following defibrillation, typically by an implanted defibrillator. The vibrational energy may be delivered following termination of the defibrillation therapy or, in some cases, may at least partly overlap the defibrillation therapy.

The vibrational transducer will be configured to apply vibrational energy to at least a portion of the heart, often including at least the ventricular regions of the heart and more typically including all regions of the heart.

The implantable vibrational transducers may be implanted at least partially over the patient's ribs or sternum, or at least partially within a gap between the patient's ribs, or at least partially under the patient's ribs, or in the abdomen. When implanted in a gap between the ribs, the gap will usually be the natural intercostal space, but in other instances could be a gap resulting from removal of one or more ribs to define the implantation space. When implanted in the abdomen, the implantable vibrational transducers may be either within or outside of the peritoneal cavity.

Delivery of the vibrational energy for the treatment of HF may comprise activating a single piezo-electric transducer, activating a piezo-composite material, sequentially activating individual vibrational transducer segments, or the like. The nature of the vibrational energy is set forth in detail below, but will usually have a frequency in the range from 0.02 to 10 MHz, a burst length less than 5,000 cycles, a burst rate less than 100 kHz, a duty cycle less than 50%, a mechanical index less than 20, and a thermal index less than 4. Usually, the vibrational energy will be delivered to at least 50% of the heart, preferably at least 75% of the heart, but alternatively may be less than 50%, of the heart.

In a second aspect of the present invention, systems for treating HF comprise a vibrational transducer and control circuitry for detecting the onset of a cardiac event, such as reduced contractility, associated with HF and for activating the vibrational transducer. The vibrational transducer is preferably implantable in a patient in the subcutaneous space near the patient's heart, and the control circuitry is adapted to cause the transducer to deliver controlled vibrational energy, usually ultrasonic energy, to the heart under conditions which promote improved cardiac function. Such conditions were described generally above in connection with the methods of the present invention.

The implantable vibrational transducer and control circuitry will usually be packaged in a common housing, but in some instances may be packaged separately in separate implantable housings, and typically connected by a cable.

The vibrational transducer may comprise any of the structures described above, and the transducer will operate under the conditions described above. The control circuitry may optionally comprise sensors such as ECG elements or other conventional circuitry for detecting cardiac events related to HF, and will usually further comprise a signal generator for the transducer, a power amplifier, and an impedance matching circuit, optionally including multiple such circuits for multi-segmented transducers. The ECG elements and circuitry are also useful for synchronizing delivery of the vibrational energy with the heart rhythm. Usually, the circuitry will further comprise a battery or a remotely rechargeable battery, such as a battery which may be recharged using inductive coupling. Usually, the control circuitry will further be adapted to communicate with an external transmitter and receiver for communications, including both patient data retrieval and programming and control of the control circuitry.

### BRIEF DESCRIPTION OF THE DRAWINGS

FIGS. 1A and 1B are schematic illustrations of a longitudinal vibrational wave traveling through biological tissue. FIG. 1A shows the pulse repetition period (PRP) while FIG. 1B shows the details of a single burst or pulse.

FIG. 2 is a schematic illustration of the effects of frequency (wavelength) on the focal characteristics of an ultrasonic beam.

FIG. 3 illustrates high frequency beams from convex, flat, and concave apertures which form divergent, mildly focused, and sharply focused beams, respectively.

FIGS. 4A and 4B illustrate the anatomy in which the vibrational transducers of the present invention are to be implanted.

FIGS. 5A-5C illustrate alternative implantation sites for the vibrational transducers and transducer assemblies of the present invention.

FIG. 6 illustrates a first embodiment of a vibrational transducer assembly constructed in accordance with the principles of the present invention.

FIG. 7 illustrates a second embodiment of a vibrational transducer assembly constructed in accordance with the principles of the present invention.

FIG. 8 illustrates a third embodiment of a vibrational transducer assembly constructed in accordance with the principles of the present invention.

FIGS. 9A and 9B illustrate a circuit configuration (FIG. 9A) and serial burst pattern (FIG. 9B) which would be suitable for operating the vibrational transducer assembly of FIG. 8.

FIG. 10 is a block diagram showing an embodiment of the control circuitry implementation of the present invention.

FIGS. 11A and B illustrate an implantation site as in FIG. 5C for the vibrational transducers and transducer assemblies of the present invention in the anterior chest.

FIG. 12 illustrates a system constructed in accordance with the principles of the present invention for chronically treating a patient in order to promote long-term improvement in heart function.

FIG. 13 illustrates a system constructed in accordance with the principles of the present invention for treating acute cardiac events associated with heart failure.

FIG. 14 illustrates a system constructed in accordance with the principles of the present invention for permitting the patient or other individual to manually initiate vibrational treatment to improve heart function.

FIG. 15 illustrates a system constructed in accordance with the principles of the present invention for automatically delivering vibrational energy to improve heart function following treatment with an implantable defibrillator.

### DETAILED DESCRIPTION OF THE INVENTION

The present invention relies on directing vibrational energy, particularly ultrasound energy, into cardiac tissue in order to improve cardiac function. An understanding of the nature of ultrasound energy and biological tissue is of use.

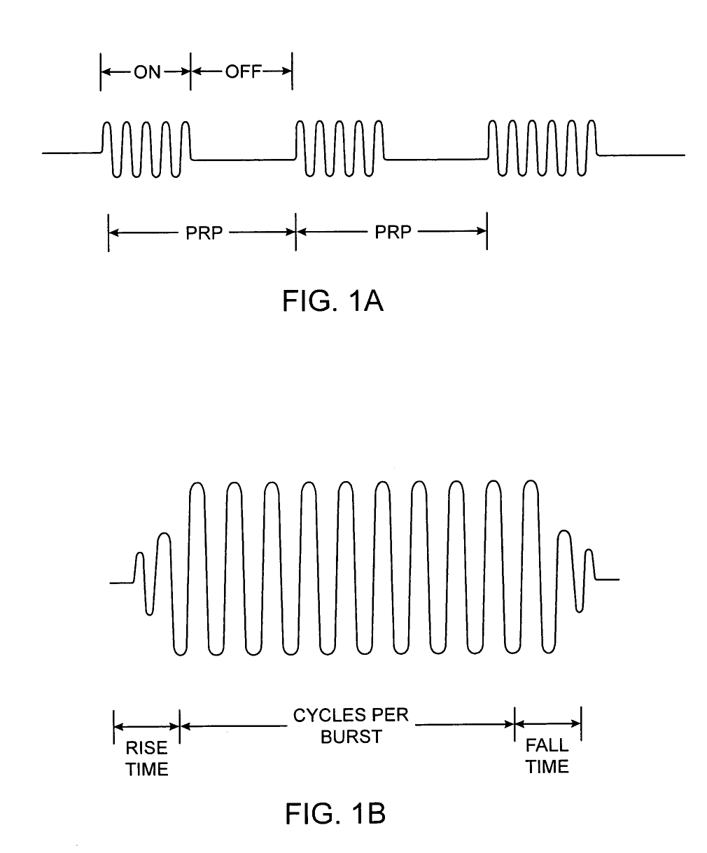

Ultrasound in biological tissues is virtually exclusively a longitudinal traveling wave, as illustrated in FIGS. 1A and 1B. The wave travels at typically 1.5 millimeters per microsecond, in a straight line unless reflected or refracted. Ultrasound may be CW (continuous wave), meaning it is on all the time, or burst mode, comprising periods of ON time separated by lengths of OFF time (FIG. 1A). The lengths of the ON and OFF periods may be the same or different, and the total of the “on time” and the “off time” is referred to as the pulse repetition period (PRP). As illustrated in FIG. 1B, ultrasound waves do not come up to peak amplitude instantaneously. The number of cycles involved in the rise time and the fall time are approximately equal to the Q (quality factor) of the device. The period of an ultrasound wave is the time for one complete cycle. The reciprocal of period is the frequency. Bursts may occur at any selected frequency. The burst rate is defined as the pulse repetition frequency (PRF), which is the reciprocal of the pulse repetition period (1/PRP). The amplitude of the wave can be defined in terms of pressure. In power applications, the magnitude of peak positive pressure is usually greater than that of the peak negative pressure. The waveform is slightly asymmetric due to non-linearities. These non-linearities arise from different velocities of sound in the body as a function of signal strength, and are dependent on the distance of travel through tissue and of course, amplitude.

From the above basic descriptors, other ultrasound parameters follow. The duty cycle is defined as the percent of time the ultrasound is in the ON state. Thus, a continuous wave would have a duty cycle of 100 percent. Intensity is the ultrasound power per unit area. Further common definitions are Ispta (intensity, spatial peak temporal average), the average intensity in the center of the beam over all time, and Isppa (intensity, spatial peak pulse average), the average intensity in the center of the beam averaged only over the duration of the pulse or during the ON state.

Two additional parameters are the Mechanical Index (MI) and the Thermal Index (TI). MI is defined as the peak negative pressure in units of MPa divided by the square root of frequency in units of MHz. The parameter is defined for diagnostic ultrasound and reflects the ability of ultrasound to cause mechanical damage, across a wide range of frequencies. The FDA guideline for diagnostic ultrasound allows a maximum MI=1.9. TI for soft tissues is defined as the average power in the beam in milliwatts times the frequency in MHz divided by 210. TI defines the capability of ultrasound to create thermal bioeffects in tissue, and a value of unity corresponds to a theoretical temperature rise in normal tissue of one degree Centigrade. These expressions show important trends for ultrasound. For a given pressure, lower frequencies tend to result in greater mechanical bioeffects. Further, for higher frequencies, there is a stronger tendency for greater thermal bioeffects.

An ultrasound beam is attenuated by the tissues through which it propagates. Tissue motion has no effect on ultrasound attenuation. At frequencies below 5 MHz, attenuation in blood is negligible. Attenuation in myocardium, muscle, fat, and skin is approximately 0.3 dB per MHz per centimeter of propagation path. Consequently, a 1 MHz beam will suffer little attenuation through the body wall and heart. All frequencies of ultrasound do not propagate well through air; it is virtually totally attenuated. The lungs and bowel gas essentially totally obstruct the beam. Attenuation in bone is strongly frequency-dependent. The attenuation at 1 MHz is in excess of 12 dB, rising almost linearly with frequency. At 100 kHz, attenuation is negligible.

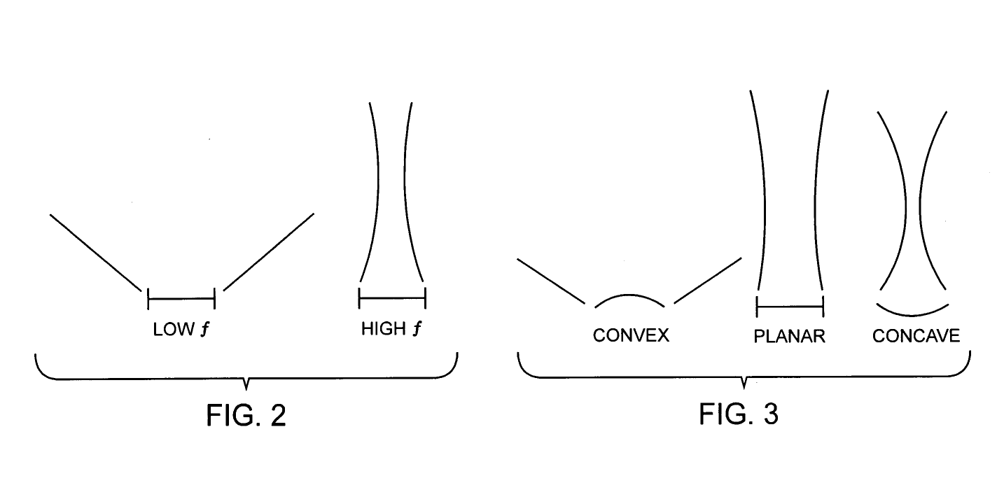

Ultrasonic beams are highly dependent on the aperture of the radiator and the frequency, and whether the beam is continuous wave or burst mode. A simple rule is that in the far field, the beam width is given by the wavelength divided by the aperture. Given the same sized apertures, a low frequency (Low f) beam might be almost isotropic (equal intensity in all directions) while a high frequency (High f) beam will be focused, as illustrated in FIG. 2. Further, the shape of the aperture will affect the beam. FIG. 3 depicts high frequency beams from convex, planar, and concave apertures, forming divergent, mildly focused, and sharply focused beams, respectively. In the far field, pulsed and continuous beams have approximately the same profiles. In the near field, however, continuous beams are characterized by multiple peaks and valleys due to constructive and destructive interference, respectively, of wavefronts from across the aperture. (For short bursts of ultrasound, constructive and destructive interference is limited to emissions from smaller portions of the aperture, and consequently, near field emission profiles are more uniform.)

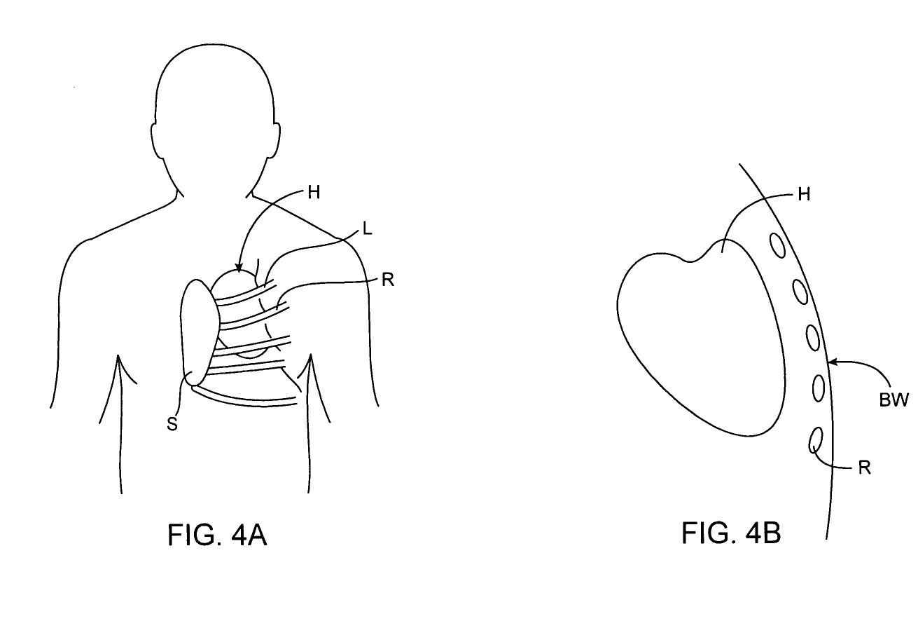

Referring now to FIGS. 4A and 4B, the present invention relies on directing ultrasound and other vibrational energy to regions of the heart H in order to stabilize cardiac function and/or treat an acute event associated with HF as generally discussed above. In particular, it may be desirable to be able to direct the ultrasonic energy over either a region of the heart or as great a portion of the heart as possible in order to assure maximum effectiveness. Usually, the present invention will provide for directing the ultrasonic energy to at least 50% of the heart, preferably at least 75%. Alternatively, it may be desirable to direct the ultrasonic energy to specific regions of the heart having poor function, covering less than 50%, or preferably less than 25% of the heart. As the heart is located beneath the body wall (BW), ribs R and sternum S, however, the vibrational transducer assembly (as described in greater detail below) must be properly located to deliver the energy. Bone and cartilage significantly attenuate the propagation of high frequency ultrasonic energy, and the lungs L (which are filled with air) will totally obstruct the transmission of such energy.

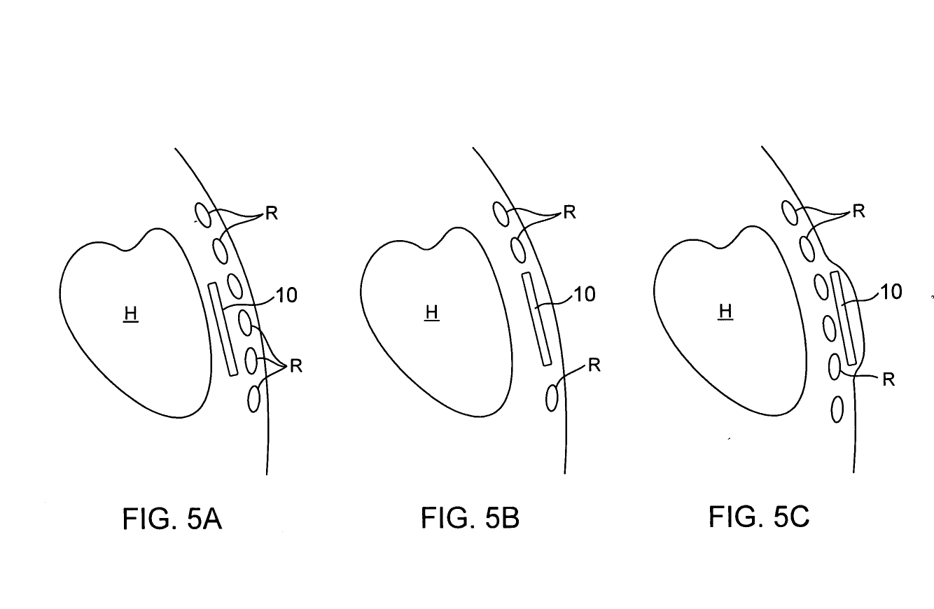

It will generally be preferred to implant a vibrational transducer assembly 10 either over the ribs R and/or sternum, as shown in FIG. 5C, between or in place of the ribs, as shown in FIG. 5B, or perhaps less desirably under the ribs R, as shown in FIG. 5A. When implanted beneath the ribs R, the vibrational transducer assembly 10 will usually be placed over or spaced slightly anteriorly from the pericardium. Alternatively, but not shown, the transducer assembly may be implanted in the abdomen, either within or outside of the peritoneal cavity.

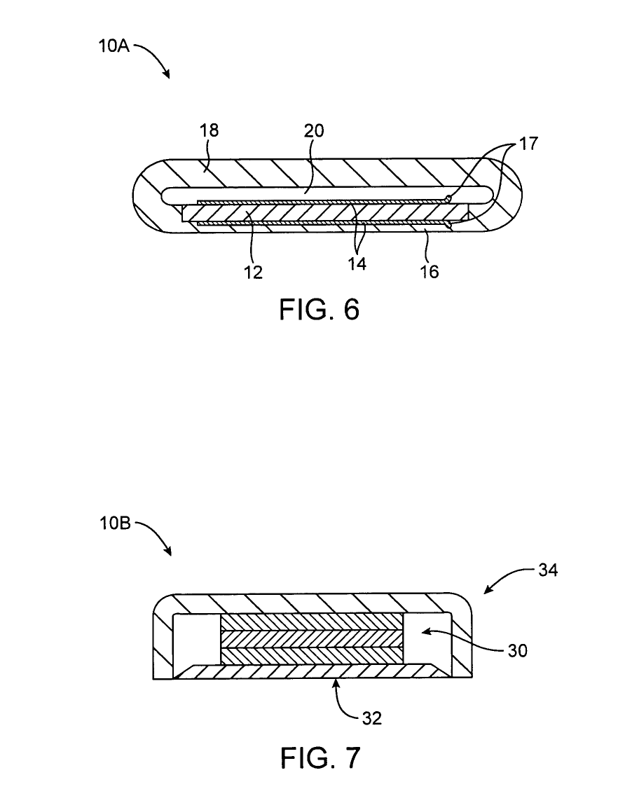

Referring now to FIG. 6, a first exemplary vibrational transducer assembly 10A comprises a quarter wave front surface matched device. A half-wave thickness of piezo-electric ceramic 12 is sandwiched between thin layer electrodes 14 having leads 17 and a quarter-wave matching layer 16 disposed over the first surface. The piezo-electric ceramic 12 is positioned in a housing 18 with an air cavity 20 at its rear surface. In this way, the quarter-wave matching layer 16 provides a front surface of the assembly 10A, and the edges and back of the housing need only be strong enough to provide mechanical support. The air cavity 20 will typically have a width of about 1 mm, and the thickness of the ceramic and matching layer will vary depending on the desired frequency of operation. Table 1 below shows the operational frequencies and thicknesses of the ceramic layer 12 and matching layer 16.

TABLE 1

| Device Frequency | Ceramic | Matching Thickness |
|---|---|---|
| (MHz) | Thickness (mm) | (mm) |
| 2.0 | 1.0 | 0.37 |
| 1.0 | 2.0 | 0.75 |
| 0.5 | 4.0 | 1.5 |
| 0.25 | 8.0 | 3.0 |
| 0.10 | 20.0 | 7.5 |

The methods of the present invention likely result from the mechanical effects of ultrasound. As such, the maximum frequency might be on the order of 1 MHz. From a structural point of view, at 0.10 MHz, the device package thickness might be on the order of 30 mm thick, probably the maximum acceptable for an implant. If the device needs to be implanted over the ribs, or placed externally, the low frequencies are preferred. At 0.25 MHz, the attenuation due to bone might be minimal, thus suggesting an operational frequency in the 0.10 to 0.5 MHz range.

Operating below 0.25 MHz with a conventional quarter wave device may not be especially advantageous due to the higher voltages needed to drive the device. Also, as the device gets thicker, it becomes substantially heavier.

As shown, the transducer assembly 10A may be substituted with a 1-3 piezo-composite material instead of the piezo-electric ceramic. Piezo-composite material consists of piezo-electric ceramic posts in a polymer matrix. Such materials are thinner than the equivalent pure ceramic material needed to achieve a particular frequency and there is no need to provide a matching layer. Thus, a simple seal providing electrical insulation may be substituted for the matching layer 16 of FIG. 6. Suitable thicknesses for the piezo-composite material are shown in Table 2 below.

TABLE 2

| Device Frequency | Piezo-composite |
|---|---|
| (MHz) | Thickness (mm) |
| 2.0 | 0.75 |
| 1.0 | 1.5 |
| 0.5 | 3.0 |
| 0.25 | 6.0 |
| 0.10 | 15.0 |

Besides creating a thinner package, the piezo-composite materials have another significant benefit in that they can be easily curved, potentially to conform to anatomical features or to optimize the transducer beam profile. It must be remembered that any curvature will affect the focal characteristics of the device.

Yet further, as shown, the transducer assembly 10A may be substituted with recently developed higher strain materials such as single crystal or polymer piezo-electrics instead of the piezo-electric ceramic. The single crystal materials would utilize a similar structure as depicted in FIG. 6. Polymer piezo-electric materials may be backed by a rigid foam material and would utilize a layer of high voltage insulator over the front surface instead of a quarter-wave matching layer. Alternatively, the polymer piezo-electric material may be backed with a high impedance material. Both backing techniques facilitate the projection of the maximum amount of energy into the patient.

Driving materials for transducers may also include any other electromechanical material, a specific example being magnetostrictive materials.

Referring now to FIG. 7, a vibrational transducer assembly 10B may be formed as a variation on a Tonpilz transducer where a piezo drive 30 (shown as a stack of piezo-electric material) induces ultrasonic vibration in a front vibrator 32. The package 34 provides the necessary tail mass for operation of the transducer assembly. Optionally, a structure (not shown) for retaining the front surface vibrator 32 against the ceramic stack 30 and housing 34 may be provided. Strong vibrations of the surface vibrator may exceed the tensile strength of the ceramic and/or bonding material. Such transducer assemblies are particularly well suited to operation at low frequencies, 0.1 MHz and below.

The device of the present invention may require an aperture generating a relatively wide acoustic beam in order to deliver ultrasonic or other vibrational energy over a relatively large portion of the heart. Due to biological constraints, the transducer may be in close proximity to the heart, and as such, the heart will be in the near field of the acoustic beam. With typical human heart dimensions of 12 cm in length and 10 cm in width, the ultrasonic or other vibrational energy aperture will typically be circular with a diameter on the order of 10 cm, more preferably elliptical with long and short axes of 12 and 10 cm, respectively, and most preferably elliptical with the ultrasonic or other vibrational energy aperture slightly exceeding the dimensions of the heart to assure maximal coverage of myocardium with therapeutic energy. It is recognized that many different sizes of devices might be required to meet the needs of different sized patients.

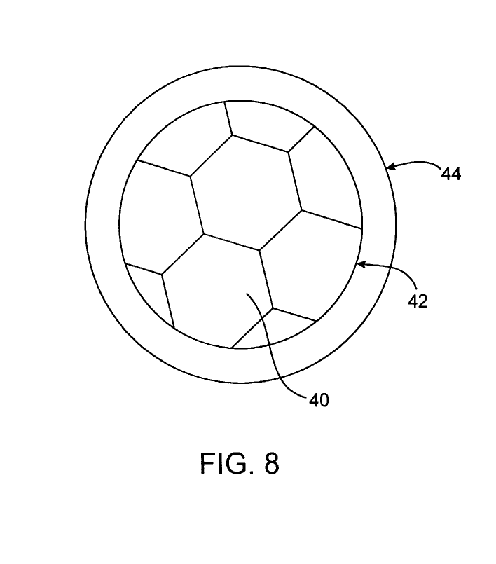

Further variations on device design are possible. Specifically, in the case of the single crystals, current technology does not provide material with dimensions consistent with the sizes projected to cover a significant fraction of the heart. Consequently, a mosaic structure of individual pieces or sections 40 of piezo electric material, as depicted in FIG. 8 might be employed. The sections 40 are arranged within an ultrasonic radiative aperture 42 in a casing 44. The sizes of individual pieces would be consistent with current manufacturing technology, currently approximately one inch on the side. The individual crystals may be wired in parallel and be driven by a single signal generator, power amplifier, and impedance matching circuit. Alternatively, the single crystals may have individual signal generators, driving amplifiers, and/or impedance matching circuits for parallel or serial operation. Alternatively, the single crystals may be driven in a sequential (multiplexed) manner by a single signal generator, power amplifier, and matching circuit.

All of the alternative devices may be driven with a high voltage and a high current. After appropriate electrical impedance matching, the current drain on the battery may exceed the capability of the same. It is thus proposed to segment the aperture into multiple individual pieces of piezo-electric, as depicted in FIG. 8 and as described above. In this case, each element may be driven by an individual power amplifier, impedance matching circuit, and signal generator (or a signal generator gated to individual amplifiers). Alternatively, the single crystals may be driven in a sequential (multiplexed) manner by a single signal generator, power amplifier, and matching circuit. As such then, exposure of the heart would be segmental. If, for example, the aperture consisted of 10 elements, operating with 5 cycles at 1 MHz, each element might be triggered every 50 microseconds, allowing for an effective 10 percent duty cycle. This would reduce the peak current demand on the battery by a factor of 10.

The ultrasonic transducer may also be structured as a two-dimensional phased array or as an annular array to achieve specific requirements related to the delivery of ultrasonic power uniformly throughout the heart. In this case, each element would be driven individually such that the combination of elements produces a sharp or broad beam in a particular direction. Alternatively, each element may be driven in serial format to generate a roster of individual beams with wider profiles.

The mosaic of individual pieces may be mounted on a flat coplanar surface, or the devices might be so mounted as to give the front surface of the device either a concave or convex surface for better implantation under the patient's skin.

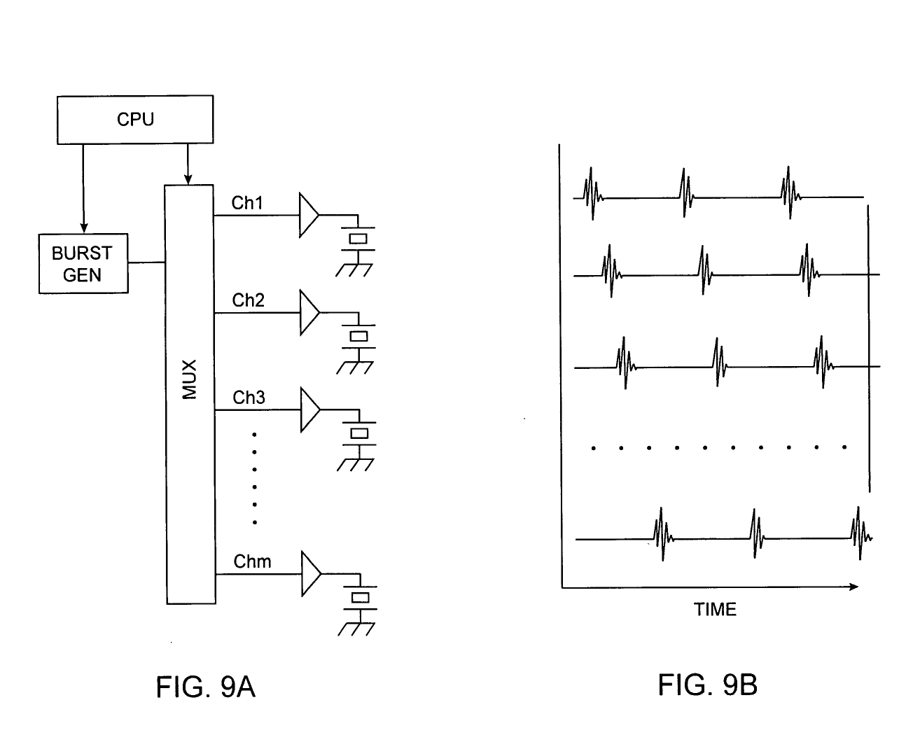

FIGS. 9A and 9B depict one possible circuit configuration for generating serial bursts from the segmented aperture, and further depicts the interlaced output from each of the individual elements within the aperture. It is possible to generate multiple bursts from every element during a small fraction of the cardiac cycle. The myocardium will effectively experience simultaneous ultrasound exposure. Care must be exercised in the implementation of this concept to prevent excessive beam spreading from the smaller elements and loss of far field signal strength. Low frequency devices would be more prone to this problem than high frequency devices.

Alternatively, the segmented aperture of individual elements of electro mechanical material, or clusters of one to several posts of a piezo composite material, may be driven in a phased sequence, so as to create an ultrasound beam in one of several particular directions. “Phasing” means that the driving signals applied to all elements or segments of the aperture have such time delays that the wavefronts from each element or segment arrive at a designated tissue mass at the same time (constructive interference). Although the amplitude in this tissue mass will be greater due to the focusing effect of the phased aperture, the beam may no longer cover the entire region of tissue requiring treatment. Consequently, in rapid succession, on time scales very small compared to the time of the cardiac cycle, the beam may be directed to multiple tissue masses in the region of treatment, so as to effectively uniformly expose the entire region with ultrasound.

Circuit configurations for operation in a phased array mode may be quite similar to the circuit configuration depicted in FIG. 9A. For phased array operation, all elements would be operative at the same time, albeit with different time delays. The burst generator would provide the different time delays which would be directed to specific amplifiers/elements through the multiplexer (MUX). Multiple sets of time delays would result in beams in multiple directions.

Instead of segmenting the aperture in a compact two-dimensional format, the aperture may be comprised of a series of segments or elements in a linear arrangement. Such an array of elements may be implanted or fixed externally for directing vibrational energy to the heart from between the ribs. Indeed, a second string of elements could be implemented in similar format, for directing vibrational energy to the heart through another intercostal space, either above or below the first string of elements. Alternatively or in conjunction, a string of elements may be implemented over the sternum. Although there will be some attenuation of the ultrasonic beam, directing vibrational energy through the sternum will assure a pathway to the heart unimpeded by lung tissue. The single or multiple linear strands of aperture segments or elements can be electrically driven in parallel or in serial format, or driven in a phased format for targeting of a specific region of the heart, or for sweeping the ultrasonic beam across a greater portion of the heart.

For therapy directed to specific regions of the heart, the device of the present invention may not require an aperture for generating a wide acoustic beam since it is not necessary for the acoustic beam to deliver energy to the majority of the heart. Thus, pacing may be accomplished by delivering vibrational energy from a portion of the transducer aperture using a segmental design, or alternatively, from a separate transducer aperture generating a narrower acoustic beam. If using a separate transducer, the separate transducer may be smaller in size and of a different shape. Thus, the invention may be comprised of one or more that one transducer assembly, connected by a cable (not illustrated).

It is assumed that the desired effect is a mechanical effect. Operating a transducer in continuous wave mode creates a maximum thermal effect and a minimal mechanical effect. Operating in a burst mode with a low duty cycle and a high amplitude minimizes thermal effects and maximizes mechanical effects. It is further believed, with some empirical evidence, that high burst rates (and short burst lengths) provide the yet further enhancements to a mechanical effect. Consequently, a preferred design will be for shortest possible burst lengths, maximum amplitude, and duty cycle to the thermal limit.

The above paragraphs discussed some of the packaging considerations for the device. To summarize, the overhead on the aperture is expected to be minimal, perhaps adding 5 to 10 mm to the diameter of a device. The thickness of the device will be defined by the type and the frequency. The electronics package (and battery) can be combined with the transducer or can be separately housed, with a cable between the two units.

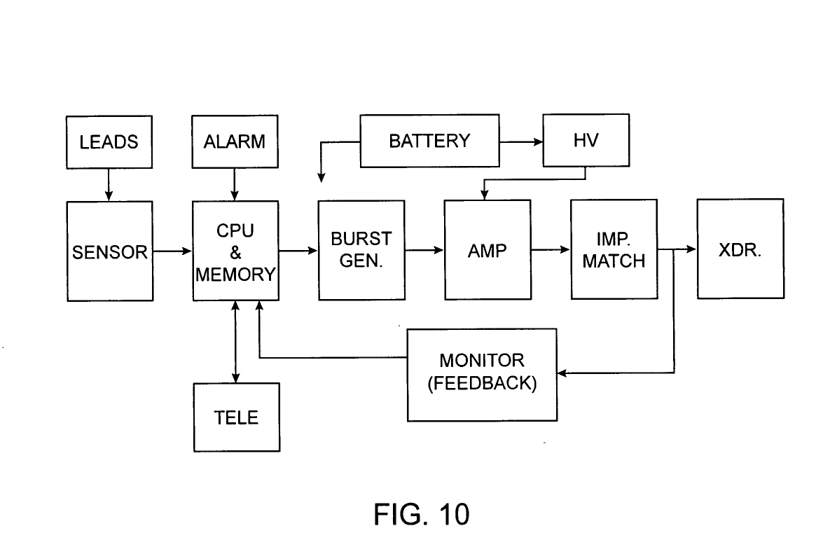

FIG. 10 represents a block diagram of a possible electronics package. The sensor circuit would be monitoring the heart and the power side of the system would generally remain idle until a specified time interval has elapsed or after a cardiac event has occurred. The sensor circuits may be integral with the CPU. Once the time interval has elapsed or the event is detected, the CPU would trigger the burst generator which would generate a preprogrammed series of bursts, for a specified period of time or until sensor monitoring indicates that heart function has returned to an acceptable level. The electrical bursts would pass to a power amplifier, an impedance matching circuit, and on to the transducer. A battery would supply power for the typically digital circuits in the CPU, telemetry, sensor, and burst generator, the typically analog circuits in the front ends of the sensor and amplifier, and to a voltage converter producing the high voltage for the output stages of the amplifier. Monitoring circuitry would provide feedback to the CPU about the actual performance of the power amplifier and transducer(s).

A battery volume on the order of one or two commercial “D” cells is anticipated. The amplifier and impedance matching circuits might require on the order of 25 cubic centimeters of volume, and the digital portions on the order of 5 cubic centimeters. In all, it is reasonable to assume that the package could be implanted into the chest of a human. Use of a rechargeable battery system utilizing transcutaneous inductive energy transmission or other charging apparatus may be beneficial.

The circuitry of FIG. 10 may be adapted to drive the associated vibrational transducer under conditions which will impart vibrational energy to the heart so that HF is abated. In particular, the vibrational transducer may be operated under the conditions specified in Table 3. The device of the present invention may or may not allow for synchronization of the therapeutic ultrasound or vibrational energy burst to the cardiac cycle. In a first embodiment, once a heart function abnormality is detected, the system will immediately initiate the preprogrammed therapeutic protocol, irrespective of the time point on the cardiac cycle. In a second embodiment, the system may trigger during any time within specified intervals of the cardiac cycle. In yet a third possible embodiment, the system may trigger treatment during a specific portion of the cardiac cycle, for example, during the refractory period. The refractory period is defined as that portion of the cardiac cycle in which the heart tissue is not excitable.

It is anticipated that the vibrational therapy might be applied for the complete cardiac cycle or a portion thereof. It is further anticipated that the vibrational energy therapy might be repeated for more than one cardiac cycle.

TABLE 3

| | Preferred | More preferred | Most preferred |
|---|---|---|---|
| Parameter | Implementation | Implementation | Implementation |
| Frequency (MHz) | 0.020-10.0 | 0.050-1.00 | 0.100-0.300 |
| Burst length (cycles) | <5000 | <500 | <10 |
| Burst rate (Hz) | >10 | >300 | >1000 |
| Duty cycle (%) | <50 | <10 | <2 |
| No. of cardiac cycles | as required | <5 | 1 |
| Portion of | 100% | <50% | <10% |
| cardiac cycle | | | |
| MI | <20 | <10 | <5 |
| TI | <4 | <1 | <.1 |
| Cardiac cycles from | <10 | <5 | <2 |
| sense to trigger | | | |

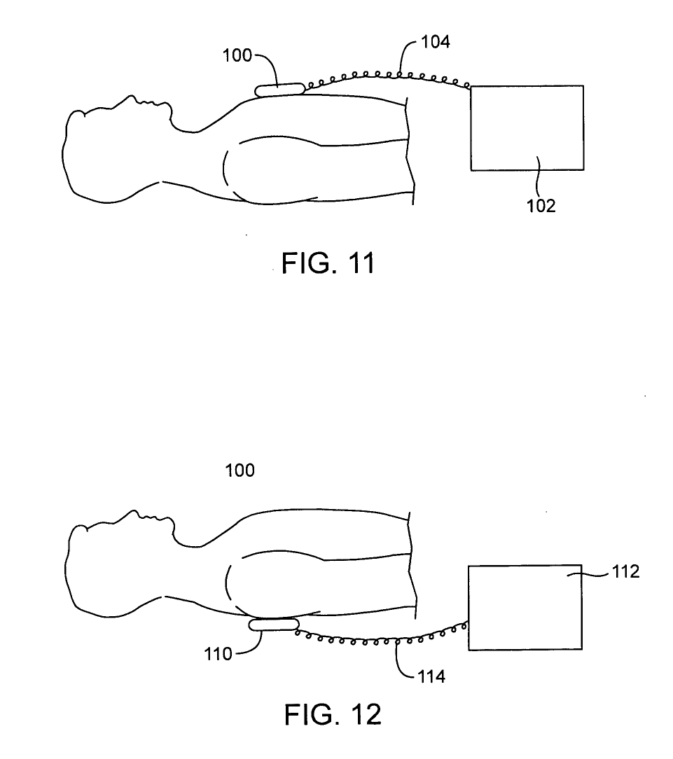

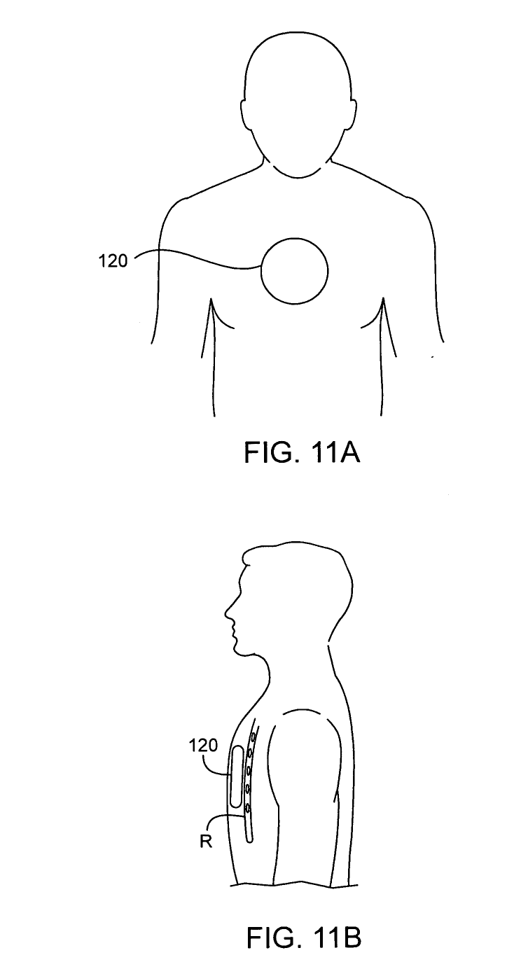

The device designs and implementations referred to thus far are generally useful for HF treatment. The treatment of HF, however, may be accomplished with systems which may be somewhat simpler than those described above and which may be deployed at body locations in addition to those described above. In particular, the vibrational transducers may be adapted for manual control by either the patient or by a doctor or other medical personnel. Treatment of HF may be accomplished with implanted vibrational transducers, with both automatically triggered and manually triggered modalities. The circuitry for automatic triggering of transducers has been discussed above. Manual triggering may be accomplished using an external wand, such as a radio frequency or magnetic controller, in order to initiate operation of the transducer. For example, an implantable transducer 120 may be placed subcutaneously in an area of the anterior chest directly over the ribs and/or sternum and preferably over the ventricular region of the heart, as shown in FIGS. 11A and 11B.

Most simply, the vibrational transducers may be incorporated into external units capable of being applied to the anterior chest. Such units will both provide for acute treatment and enable the determination of patients in whom a subsequent implantable system will be beneficial. For placement, the patient will usually be reclining on the table, bed, or ground; vibrational transducer attached to an external generator by an attached cord is applied over the patient's chest, preferably using a gel layer to enhance contact. Usually, the transducer will be placed generally over the heart and the transducer may be configured to direct the energy over a specific region, preferably the ventricular region.

In the manually controlled embodiments of the vibrational transducers, circuitry for sensing the electrocardiogram will usually be included in order to synchronize the timing of the delivery of the vibrational energy to an appropriate point in the cardiac cycle based on detection of the ventricular QRS.

The vibrational transducer systems described above will be combined with suitable circuitry and other components in order to permit actuation of the transducer(s) under particular conditions and in response to particular events, depending on the desired treatment protocol to be implemented. In general, the vibrational transducer systems may function automatically in response to detected conditions or may be activated manually by an external activator, such as a patient “wand” or radiofrequency remote control which permits the patient to initiate function of the transducer whenever desired.

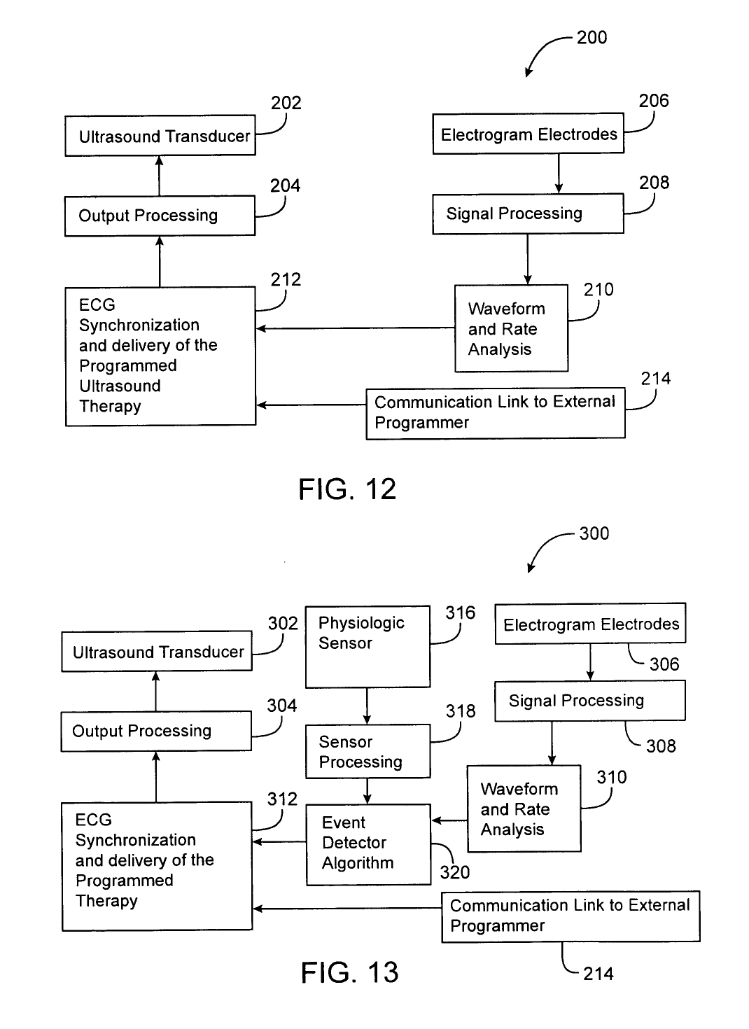

Referring in particular to FIG. 12, a system 200 constructed in accordance with the principles of the present invention for allowing chronic treatment of the heart in order to provide long-term benefit is illustrated. The system comprises an ultrasound transducer 202, as generally described above. At a minimum, the system 200 will include processing circuitry 204 which will control the timing and duration of transducer operation. Under the simplest protocols, the ultrasound energy may be delivered continuously or under a simple timed program. Preferably, however, the vibrational energy will be delivered in a manner synchronized with a portion of the cardiac cycle in order to avoid undesirable arrhythmic effects. In such cases, the system includes electrodes 206 implanted to detect heart function, signal processing circuitry 208, waveform and rate analysis circuitry 210, and circuitry 212 for synchronizing operation of the transducer with the detected ECG. The system will be adapted to permit external communication in order to reprogram the system, retrieve patient data, and the like.

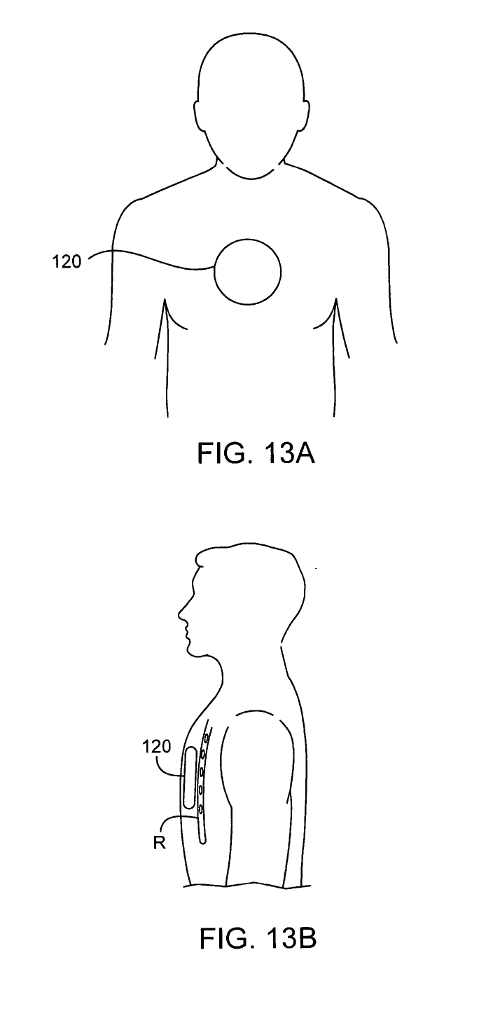

Referring now to FIG. 13, a system 300 is depicted including physiologic sensors 316 for detecting cardiac events, such as low contractility, chamber enlargement, pressure excursion, and the like. System 300 includes transducer 302, output processing circuitry 304, ECG synchronization circuitry 312, electrodes 306, signal processing circuitry 308, and waveform and rate analysis circuitry 310, generally as described above for system 200. Data from the physiologic sensor 316 is fed to sensor processing circuitry 318 which in turn is delivered to circuitry 320 which is programmed to detect the cardiac event to be treated. When such an event is detected, the signal is sent to the ECG synchronization circuitry in order to initiate ultrasound function. Typically, the system 300 will also include a communications link 214 capable of wireless communication in order to reprogram the system, retrieve patient data, and the like.

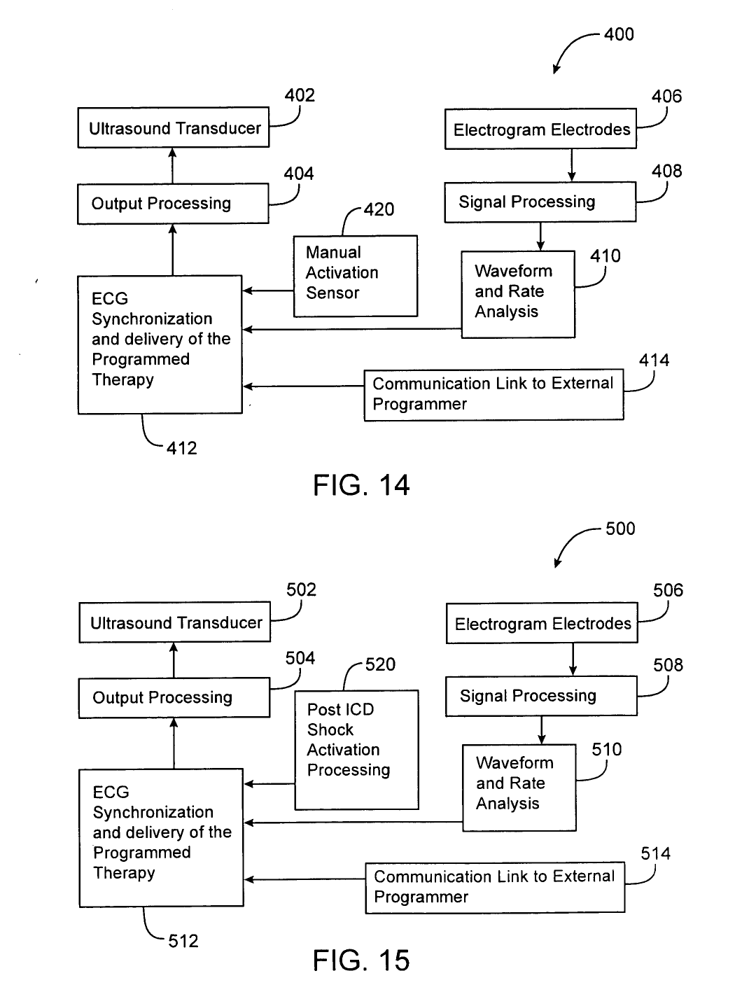

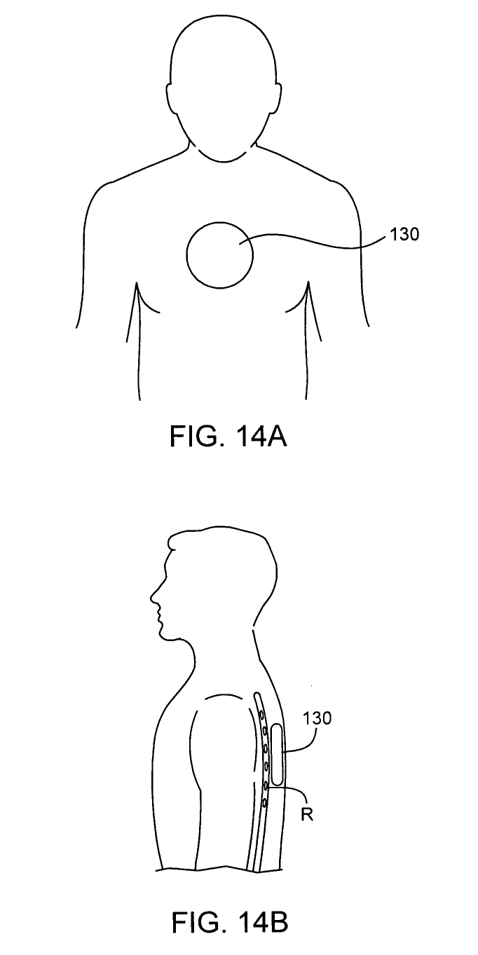

Referring now to FIG. 14, a system 400 which permits manual activation of the ultrasound transducer 402 is illustrated. The system 400 includes output processing circuitry 404, ECG synchronization circuitry 412, electrodes 406, signal processing circuitry 408, and waveform and rate analysis circuitry 410, as generally described with the prior systems. A manual activation sensor 420 is further provided in order to permit the user or other individual to selectively initiate output of the ultrasound transducer whenever desired, typically when the patient senses a cardiac event. Usually, external circuitry 414 will be provided to permit external reprogramming of the system.

Referring now to FIG. 15, a system 500 for initiating ultrasound transducer operation after or overlapping with operation of an implantable cardiac defibrillator (ICD) includes an ultrasound transducer 502, output processing circuitry 504, ECG synchronization circuitry 512, ECG electrodes 506, signal processing circuitry 508, and waveform and rate analysis circuitry 510, as generally described above with the prior systems. Implantable circuitry 520 is further provided and coupled to an implantable defibrillator in order to detect operation of the ICD; alternatively, the circuitry 520 would remotely sense the operation of the ICD. The circuitry 520, once defibrillator actuation is detected, will initiate operation of the ultrasound transducer in order to deliver vibrational energy to the heart. Typically, the vibrational transducer operation will be initiated immediately following discharge of the ICD. It is possible, however, that the ultrasound transducer operation could be initiated to briefly overlap with the firing of the ICD. As with prior systems, an external link is preferably provided in order to permit external reprogramming of the system. In some instances, the HF systems of the present invention may be combined with an ICD (or other implantable therapeutic and/or diagnostic device) in a common enclosure, optionally sharing a power supply, communications circuitry and/or other common features.

While the above is a complete description of the preferred embodiments of the invention, various alternatives, modifications, and equivalents may be used. Therefore, the above description should not be taken as limiting the scope of the invention which is defined by the appended claims.

1. A method for treating heart failure, said method comprising: delivering vibrational energy from a vibrational transducer to a heart in a patient suffering from or at risk of heart failure.

2. A method as in claim 1, wherein delivery is performed by an implanted vibrational transducer.

3. A method as in claim 1, wherein delivery is performed with an external vibrational transducer.

4. A method as in any one of claims 1-3, wherein the vibrational energy is delivered under conditions which increase at least one of contractility, vasodilation, tissue perfusion or cardiac output.

5. A method as in any one of claims 1-3, wherein the vibrational energy is delivered substantially continually.

6. A method as in any one of claims 1-3, wherein the vibrational energy is delivered in response to a manually initiated external signal.

7. A method as in any one of claims 1-3, wherein the vibrational energy is selectively delivered following defibrillation.

8. A method as in any one of claims 1-3, wherein the vibrational energy is delivered in response to detection of a cardiac event.

9. A method as in claim 8, wherein the patient or another individual detects the cardiac event and initiates delivery of the vibrational energy.

10. A method as in claim 8, wherein detection is performed by an implanted sensor, which automatically initiates delivery of the vibrational energy.

11. A method as in any one of claims 1-3, further comprising diagnosing the patient to be suffering from or at risk of heart failure.

12. A method as in any one of claims 1-3, wherein the vibrational energy is delivered to substantially the entire heart.

13. A method as in any one of claims 1-3, wherein the energy is delivered preferentially to a ventricular region of the heart.

14. A method as in any one of claims 1-3, wherein the vibrational transducer is implanted at least partially under the patient's ribs.

15. A method as in any one of claims 1-3, wherein the vibrational transducer is implanted at least partially in a gap between the patient's ribs.

16. A method as in any one of claims 1-3, wherein the vibrational transducer is implanted at least partially over the patient's ribs.

17. A method as in any one of claims 1-3, wherein the vibrational transducer is implanted in the abdominal region.

18. A method as in any one of claims 1-3, wherein the vibrational transducer is implanted in a subcutaneous space of the anterior chest over the sternum.

19. A method as in any one of claims 1-3, wherein the vibrational transducer is implanted in a subcutaneous space of the anterior chest over the ribs.

20. A method as in any one of claims 1-3, wherein the vibrational transducer consists essentially of a single piezo-electric ceramic in a housing with an air backing.

21. A method as in any one of claims 1-3, wherein the vibrational transducer comprises a piezo-composite material including piezo-electric ceramic posts in a polymer matrix.

22. A method as in any one of claims 1-3, wherein the vibrational transducer comprises single crystal piezo-electric, polymer piezo-electric, or magnetostrictive materials.

23. A method as in any one of claims 1-3, wherein delivering vibrational energy comprises energizing individual vibrational transducer segments either in series or parallel, wherein at least some of the segments direct vibrational energy to different regions of the heart.

24. A method as any one of claims 1-3, wherein delivering vibrational energy comprises sequentially energizing individual vibrational transducer segments, wherein at least some of the segments direct vibrational energy to the same region of the heart.

25. A method as in any one of claims 1-3, wherein the vibrational energy has a frequency in the range from 0.02 to 10 MHz, a burst length less than 5,000 cycles, a burst rate less than 100 kHz, a duty cycle less than 50%, a mechanical index less than 20, and a thermal index less than 4.

26. A method as in any one of claims 1-3, wherein the vibrational energy is delivered during a portion of the cardiac cycle.

27. A method as in claim 26, wherein the vibrational energy is delivered during the refractory period of the cardiac cycle.

28. A method as in claim 26, wherein vibrational energy delivery is timed from the onset of a cardiac cycle.

29. A system for stabilizing cardiac function, said system comprising:
a vibrational transducer implantable in a patient; and
control circuitry for detecting an onset of a cardiac event associated with heart failure and activating the vibrational transducer to deliver controlled vibrational energy to the heart under conditions which treat the heart failure.

30. A system as in claim 29, wherein the vibrational transducer is adapted to delivering vibrational energy which can increase contractility.

31. A system as in any one of claims 29 and 30, wherein the vibrational transducer is adapted to delivering vibrational energy which can increase vasodilation.

32. A system as in any one of claims 29 and 30, wherein the vibrational transducer is adapted to delivering vibrational energy which can increase tissue perfusion.

33. A system as in any one of claims 29 and 30, wherein the vibrational transducer is adapted to delivering vibrational energy which can increase cardiac output.

34. A system as in any one of claims 29 and 30, wherein the vibrational transducer and the control circuitry are packaged in a common housing.

35. A system as in any one of claims 29 and 30, wherein the vibrational transducer and the control circuitry are packaged in separately implantable housings, further comprising a cable for connecting the housings.

36. A system as in any one of claims 29 and 30, wherein the vibrational transducer consists essentially of a single piezo-electric ceramic disposed in a housing with an air backing.

37. A system as in any one of claims 29 and 30, wherein the vibrational transducer comprises a piezo-composite material including piezo-electric ceramic posts in a polymer matrix.

38. A system as in any one of claims 29 and 30, wherein the vibrational transducer comprises single crystal piezo-electric, polymer piezo-electric, or magnetostrictive materials.

39. A system as in any one of claims 29 and 30, wherein delivering comprises energizing individual vibrational segments, wherein at least some of the segments direct vibrational energy to different regions of the heart.

40. A system as in any one of claims 29 and 30, wherein the vibrational transducer comprises a plurality of separately driven segments, wherein the segments are arranged to sequentially direct vibrational energy to the same region of the heart when the system is implanted.

41. A system as in any one of claims 29 and 30, wherein the vibrational transducer is adapted to deliver vibrational energy to at least 50% of the heart when implanted.

42. A system as in any one of claims 29 and 30, wherein the vibrational transducer is adapted to deliver energy to less than 50% of the heart when implanted.

43. A system as in any one of claims 29 and 30, wherein the control circuitry drives the vibrational transducer at a frequency in the range from 0.02 to 10 MHz, a burst length less than 5,000 cycles, a burst rate less than 100 kHz, a duty cycle less than 50%, a mechanical index less than 20, and a thermal index less than 4.

44. A system as in any one of claims 29 and 30, wherein the control circuitry comprises ECG elements for detecting onset of a cardiac cycle and for timing the delivery of vibrational therapy in response to such detection.

45. A system as in claim 44, wherein the timing for the delivery of the vibrational energy is adapted to be delivered during a portion of the cardiac cycle.

46. A system as in claim 45, wherein the portion of the cardiac cycle is the refractory period of the cardiac cycle.

47. A system as in any one of claims 29 and 30, wherein the control circuitry comprises a power amplifier, an impedance matching circuit, and a signal generator, for each segment of the vibrational transducer.

48. A system as in any one of claims 29 and 30, wherein the control circuitry comprises a remotely rechargeable battery.

49. A system as in any one of claims 29 and 30, wherein the control circuitry comprises a transmitter and/or receiver for communication with an external controller.

50. A system as in any one of claims 29 and 30, wherein the control circuitry is adapted to detect cardiac events.

51. A system as in any one of claims 29 and 30, wherein the control circuitry is adapted to detect delivery of defibrillation energy.

52. A system as in any one of claims 29 and 30, wherein the system further comprises a cardiovertor defibrillator.

53. A system for stabilizing cardiac function, said system comprising:
a vibrational transducer; and
control circuitry for activating the vibrational transducer to deliver controlled vibrational energy to the heart under conditions which treat the heart failure.

54. A system as in claim 53, wherein the vibrational transducer is adapted to contact an exterior surface of the patient's skin and deliver the vibrational energy through the tissue overlying the heart.

55. A system as in claim 54, wherein the vibrational transducer is adapted to delivering vibrational energy which can increase contractility.

56. A system as in claim 55, wherein the vibrational transducer is adapted to delivering vibrational energy which can increase cardiac output.

57. A system as in any one of claims 53-56, wherein the control circuitry comprises a power amplifier, and impedance matching circuit, and a single generator, for activating the transducer.

58. A system as in any one of claims 53-56, wherein the control circuitry comprises ECG elements for detecting onset of a cardiac cycle and for timing the delivery of vibrational therapy in response to such detection.

59. A system as in any one of claims 53-56, wherein the timing for the delivery of the vibrational energy is adapted to be delivered during a portion of the cardiac cycle.

60. A system as in any one of claims 53-56, wherein the portion of the cardiac cycle is the refractory period of the cardiac cycle.

61. A system as in claim 60, wherein the control circuitry is adapted for manual delivery.

62. A system as in claim 60, wherein the control circuitry is adapted for automatic delivery in response to such detection.
